(19) United States
(12) Patent Application Publication
(10) Pub. No.: US 2026/0138942 A1
(43) Pub. Date: May 21, 2026

(54) METHOD FOR PRODUCING (METH)ACRYLIC ACID

(71) Applicant: Arkema France, Puteaux (FR)

(72) Inventors: Serge CHOUFFEUR, Saint Avold Cedex (FR); Serge TRETJAK, Saint Avold Cedex (FR); Fanny DANTON, Saint Avold Cedex (FR)

(21) Appl. No.: 19/114,845

(22) PCT Filed: Sep. 22, 2023
(86) PCT No.: PCT/FR2023/051459

§ 371 Date: Mar. 25, 2025

(30) Foreign Application Priority Data
Sep. 26, 2022 (FR) FR22.09737

(51) Int. Cl. C07C 51/44; C07C 57/04

(52) U.S. Cl. CPC C07C 51/44; C07C 57/04

(57) ABSTRACT

The present invention relates to the production of (meth)acrylic acid in a process based on the use of two distillation columns (a dehydration column and a finishing column) in the absence of an external organic solvent. The invention relates more particularly to the implementation of thermal cracking preceded by evaporation, hydrolysis of the bottom of the finishing column, followed by recycling of the gas phase of the cracker at the bottom of the dehydration column and upgrading of the residue of this cracking by hydrothermal gasification. The process according to the invention makes it possible to improve the energy balance of the process while improving the material balance.

### TECHNICAL FIELD

The present invention relates to the production of (meth)acrylic acid in a process based on the use of two distillation columns (a dehydration column and a finishing column) in the absence of an external organic solvent. The invention relates more particularly to the implementation of thermal cracking preceded by evaporation, hydrolysis of the bottom of the finishing column, followed by recycling of the gas phase of the cracker at the bottom of the dehydration column and upgrading of the residue of this cracking by hydrothermal gasification. The process according to the invention makes it possible to improve the energy balance of the process while improving the material balance.

### Technical Background and Technical Problem

The process for the synthesis of acrylic acid made use of on a large industrial scale employs a reaction for the catalytic oxidation of propylene in the presence of oxygen.

This reaction is usually carried out in the gas phase and usually in two steps: the first step carries out the substantially quantitative oxidation of propylene to an acrolein-rich mixture, and then the second step carries out the selective oxidation of the acrolein to acrylic acid.

The gas mixture resulting from the second step consists, apart from the acrylic acid, of unconverted compounds resulting from the reactants involved or of impurities generated during one at least of the reaction steps, namely:
of light compounds which are noncondensable under the temperature and pressure conditions normally employed, i.e. essentially: propylene, propane, nitrogen, unconverted oxygen, carbon monoxide and dioxide formed in small amounts by final oxidation;
of light compounds which are condensable, i.e. essentially: water, unconverted acrolein, light aldehydes such as formaldehyde, glyoxal and acetaldehyde, formic acid, acetic acid or propenoic acid;
of compounds with a boiling point slightly higher than that of acrylic acid: furfuraldehyde, benzaldehyde, maleic acid and maleic anhydride, benzoic acid, 2-butenoic acid, phenol, protoanemonin;
finally, of heavy compounds derived from the addition of compounds with nucleophilic properties on the double bond of unsaturated carbonyl monomers, by Michael reaction.

The complexity of the gas mixture obtained in this process makes it necessary to carry out a set of operations in order to recover the acrylic acid contained in this gas effluent and to convert it into a grade of acrylic acid compatible with its final use, for example the synthesis of acrylic esters or the production of polymers of acrylic acid and/or of acrylic esters.

Document EP 2 066 613, based on a “solventless” technology, describes a process for recovering acrylic acid (AA) without use of external water or of azeotropic solvent. This process employs only two distillation columns to purify the cooled gas reaction mixture: a dehydration column, and a finishing column (or purification column) fed with a portion of the bottom stream from the dehydration column.

According to this process, the cooled gas reaction stream is subjected to dehydration in a first column. The gas stream distilled at the top of the column is sent to a condenser, in which the light compounds are partly condensed and returned to the dehydration column in liquid reflux form in order to absorb the acrylic acid, the non-condensed gas effluent being at least partly returned to the reaction and the remainder being removed.

The bottom stream from the dehydration column is sent to a second column called a finishing column. During the purification/finishing step, a stream rich in heavy compounds is removed at the bottom and a distillate comprising water and light by-products is recovered at the top and condensed and then recycled at the bottom of the first dehydration column, thus forming a recirculation loop.

A stream of purified acrylic acid is recovered in the liquid or vapor form, by sidestream withdrawal from the finishing column. The acrylic acid obtained generally has a purity greater than 98.5% by weight and contains less than 0.5% by weight of water and less than 0.4% by weight of acetic acid.

The temperature and pressure operating conditions for the finishing column are not critical in this process, and can be determined in accordance with the distillation methods known from the prior art. However, preferably, the purification column is operated at a pressure below atmospheric pressure, thus preventing polymerization of the unsaturated products present, and minimizing the formation of heavy by-products. These compounds are heavy products that reduce the recovery efficiency by consuming acrylic acid monomer. In the case of an AA production unit, these are essentially:
derivatives of the addition of acrylic acid to the double bond of another acrylic acid molecule: 3-acryloxypropionic acid also known as “acrylic acid dimer” or “AA dimer”;
derivatives of the addition of acrylic acid to the double bond on an AA dimer molecule, to form the “AA trimer”, and other oligomers formed by successive additions of acrylic acid to the double bonds of the preceding AA oligomers;
derivatives of the addition of carboxylic acids, formed as by-products of the acrylic acid, or of water to the double bond of the AA or of the abovementioned oligomers.

Like radical polymerization, this covalent reaction for formation of Michael derivatives is strongly promoted by temperature. Therefore, the installation of columns with a high number of rectifying plates in order to meet the quality requirements of acrylic acid leads to disadvantages in terms of product loss, which can only be compensated for by an additional high temperature cracking treatment of the Michael derivatives to regenerate the acrylic acid monomer or recycling of this stream from the bottom of the finishing column optionally to an ester unit.

The recovery of upgradable monomers from heavy Michael derivative compounds is difficult in the case of heavy products originating from an AA production unit. Specifically, during the thermal cracking process that regenerates acrylic acid, which is distilled and upgraded, a residue remains, the viscosity of which increases greatly when high cracking efficiencies are sought, until it can no longer be extracted from the cracking reactor.

The main factor limiting the efficiency of the regeneration of the compounds derived from the Michael reaction contained in the heavy streams from AA plants is the increase in the viscosity of the heavy residue obtained at the bottom of the cracker, when the fraction rich in acrylic monomers has been vaporized, as described in document FR2727964. The evaporation of light compounds during the cracking results in a concentration of the heavy products in the residue stream and an increase in the viscosity of this stream. However, the residue has to remain sufficiently fluid after cooling to be transported and then treated for the purpose of destroying it. The viscosity of the residue obtained at the end of cracking increases with the residence time at high temperature of the mixture to be treated and with the amount of light monomers recovered by distillation. To obtain a viscosity of the residue compatible with normal transfer conditions and to reduce fouling phenomena, these two parameters must be limited, thereby having the effect of reducing the cracking yield.

Application FR2206330 complies with this improvement in the regeneration yield in a continuous process, without significant increase in the dynamic viscosity close to 1 Pa·s, by implementing hydrolysis of the heavy by-products with a weight ratio of water:heavy acrylic acid compounds ranging from 0.1 to 1.3 before implementation of thermal cracking.

In the case where there is production of light esters (methyl acrylate (MA) or ethyl acrylate (EA)) close to the AA production unit, a co-cracking of the respective heavy products can improve the situation, making the cracking residue more fluid. The proposed solution makes it possible to recover the maximum amount of AA per cracking operation while managing the viscosity of the residue formed. Thus, in document EP 717 031, it has been shown that it is possible to improve the efficiency of the recovery of these upgradable noble products if the cracking is carried out with a mixture of heavy products originating from an AA production unit and from an acrylic ester (EA) production unit, compared to the individual cracking of the heavy streams from these units. The effect of the addition of heavy products originating from the ester units (EAHP) to the heavy products originating from an AA unit (HAA) is to reduce the viscosity of the final residue. The cracking reaction is carried out with mixtures having an AA heavy products/ester heavy products ratio of 9/1 to 1/9, at a temperature of 180° C. to 220° C., under atmospheric pressure, for a residence time of 0.5 to 3 hours.

Document FR3110571 proposes combining partial condensation with the cracking reactor, thereby making it possible to increase the cracking yield without noticeable effect on the viscosity of the residue.

In the case of treating heavy acrylic acid compounds (HAA) alone, it was also envisioned to add a solvent to the residue.

Document EP 3255030 teaches the addition of higher alcohols during the cleavage of the residue, the maleic anhydride present in the residue being converted into maleic acid esters which are less sensitive to polymerization.

Document U.S. Pat. No. 6,414,183 teaches the dilution of the discharged residue with solvents such as acetic acid, water and methanol. This dissolution is carried out on a distillation column or evaporator bottom in a proportion of 0.1 to 5 times relative to the bottom product before treatment by combustion.

Application WO 2021/224044 describes a process for breaking down Michael adducts of acrylic acid, by dilution in a solvent having a boiling point at 1013 hPa of at least 170° C. and a solubility in water at 25° C. of at least 20 g per 100 g of water, said solvent being chosen from alcohols such as ethylene glycol, propylene glycol, diethylene glycol, triethylene glycol and 2-ethoxyethanol, carboxamides such as N,N-dimethylacetamide, N-methylacetamide and N,N-dimethylformamide, sulfoxides such as dimethyl sulfoxide, and sulfones such as sulfolane.

However, these solutions have several disadvantages, such as the generation of waste to be burnt, the provision of additional equipment for mixing, or energy consumption if it is assumed that water has been chosen as the solvent to be vaporized. Moreover, most of these solvents generate nitrogen-containing or sulfur-containing derivatives upon burning.

Oxidation of organic matter (incineration) to carbon dioxide and water is often used to treat organic residues and to produce heating steam. In the conventional process, rapid oxidation of organic fuels is used to produce heat, which is then transferred in a heat exchanger to a fluid such as water. A heat loss of 10-15% is expected as a result of losses necessarily occurring in the exhaust column of conventional boilers. In addition to a possible blockage due to solids supplied to the boiler, hot spots due to salt deposits on boiler tubes, or ash deposits on tube faces exposed to flame or hot gases, reduce good heat transfer and consequently heat transfer efficiency, or even result in very costly time losses due to breakage of the walls of the tubes.

Firstly, there remains a need for a solvent-free purification line for the heavy products from the bottom of the finishing column, which is independent of the operation of the (meth)acrylic ester units. Furthermore, it is desirable to be able to upgrade the final residue from the production of (meth)acrylic acid into exportable methane gas, instead of converting it into CO₂ by combustion.

### SUMMARY OF THE INVENTION

The present invention makes it possible to meet the abovementioned needs. More particularly, the invention provides an improved process for producing (meth)acrylic acid which makes it possible to better upgrade the final products usually sent for incineration, by regenerating the starting materials by cracking and by converting the residue into combustible gas. The process according to the invention makes it possible to improve the energy balance of the process while improving the material balance.

This result is obtained by implementation of thermal cracking preceded by evaporation, hydrolysis of the bottom of a finishing column followed by recycling of the gas phase of the cracker at the bottom of the dehydration column and upgrading of the residue of this cracking by hydrothermal gasification.

The subject of the present invention is a process for producing technical-grade (meth)acrylic acid in the absence of organic solvent, from a gas reaction mixture comprising (meth)acrylic acid obtained by gas-phase oxidation of a precursor of the (meth)acrylic acid, comprising the following steps:
a) a step of dehydrating said gas reaction mixture in a first distillation column called a dehydration column, resulting in a top stream, a portion at least of which is condensed and returned to the dehydration column in the reflux form, and in a bottom stream, a portion at least of which is returned to the lower part of the dehydration column to form a recirculation loop;
b) a step of distilling at least a portion of said bottom stream from the dehydration column in a second distillation column, called a finishing column, making it possible to separate a bottom stream containing heavy compounds, a top stream containing light compounds, at least a portion of which is returned to the dehydration column, and a withdrawal sidestream of technical-grade (meth)acrylic acid;
c) a step of concentrating said bottom stream from the finishing column in an evaporator resulting in a bottom stream concentrating the Michael adducts, and a top stream comprising (meth)acrylic acid which is returned to the finishing column;
d) a step of hydrolyzing said evaporator bottom stream, in the presence of water, in a hydrolyzer, resulting in the production of a stream of hydrolyzed products;
e) a step of thermally cracking said stream of hydrolyzed products in a cracker, resulting in the production of a top stream which is recycled to the dehydration column, and a bottom residue; and
f) a step of hydrothermally treating said residue, in the presence of water, carried out in hydrothermal gasification equipment, resulting in the production, at the top, of gases of methane, hydrogen and CO₂ type and, at the bottom, of solid residues and water.

The present invention makes it possible to overcome the drawbacks of the prior art. More particularly, it provides a process for obtaining a technical-grade (meth)acrylic acid of high purity having as specification a (meth)acrylic acid purity of greater than 98.5%, integrating a process for upgrading Michael adducts into reactants recycled in the process, thus increasing the productivity of the process and improving the energy balance by upgrading of the residue to be removed.

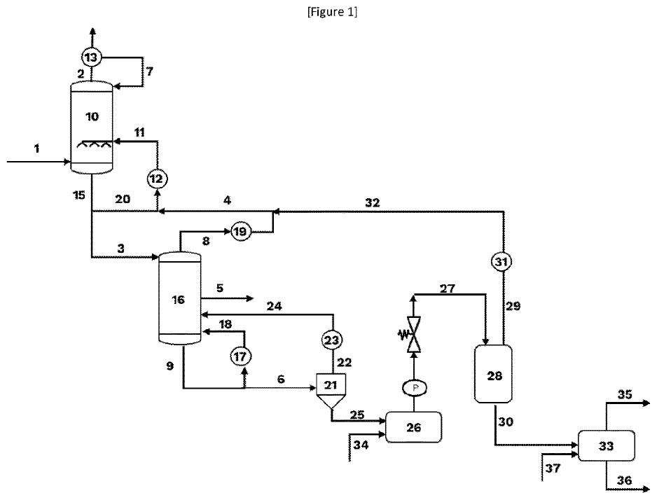

Other characteristics and advantages of the invention will become more apparent on reading the detailed description which follows, with reference to the appended FIG. 1.

FIG. 1: Overall diagram of the acrylic acid purification process with the combination of an evaporator at the bottom of the finishing column, and a thermal cracker preceded by a hydrolyzer, with hydrothermal gasification equipment.

### DETAILED DESCRIPTION OF THE INVENTION

The invention provides an improved process for obtaining a technical-grade (meth)acrylic acid of high purity.

According to various implementations, said process comprises the following features, where appropriate in combination.

According to one embodiment, the process according to the invention is a process for producing technical-grade acrylic acid of high purity.

According to one embodiment, the process according to the invention is a process for producing technical-grade methacrylic acid of high purity.

The invention is described below by taking the example of the process for producing acrylic acid. The process according to the invention may also comprise other preliminary, intermediate or subsequent steps provided that they do not negatively affect the production of purified acrylic acid.

According to one embodiment of the invention, the acrylic acid precursor is acrolein.

According to one embodiment of the invention, the acrolein is obtained by oxidation of propylene or by oxydehydrogenation of propane.

According to one embodiment of the invention, the gas reaction mixture comprising acrylic acid obtained by oxidation in the gas phase of an acrylic acid precursor comprises carbon of renewable origin.

According to one embodiment of the invention, the acrylic acid precursor is derived from glycerol, from 3-hydroxypropionic acid or from 2-hydroxypropionic acid (lactic acid).

According to a preferred embodiment of the invention, the gas reaction mixture comprises acrylic acid derived from propylene obtained according to a two-step oxidation process.

According to one embodiment, the finishing column is a conventional distillation column.

According to one embodiment, the finishing column is a dividing wall column.

According to one embodiment, the finishing column operates under a reduced pressure of 5 to 60 kPa.

According to one embodiment, an aldehyde reducing chemical may be injected into the feed of the finishing column.

According to one embodiment, the evaporator placed at the bottom of the finishing column is a film evaporator working at a reduced pressure of 0.5 to 100 kPa.

According to one embodiment, the product from the top of the evaporator is recycled into the sidestream withdrawal line of the finishing column.

According to one embodiment, the product from the top of the evaporator is recycled to the bottom of the finishing column, below the sidestream withdrawal line.

According to one embodiment, the pressure in the hydrolyzer varies between 0.1 and 2 MPa, preferably between 0.5 and 1.5 MPa.

According to one embodiment, the water/adducts weight ratio in the hydrolyzer ranges from 0.1 to 1.3, limits included.

According to one embodiment, the temperature in the hydrolyzer varies between 80° C. and 200° C., preferably between 150° C. and 200° C.

According to one embodiment, the thermal cracking reaction takes place in the absence of catalyst.

According to one embodiment, the cracking temperature is between 140° C. and 260° C., preferably between 160° C. and 210° C.

According to one embodiment, the thermal cracking is performed on acrylic acid adducts.

According to one embodiment, the thermal cracking is performed on a mixture of adducts of acrylic acid and esters.

According to one embodiment, the residence time of the reaction mixture in the cracking reactor is between 0.5 h and 10 h, preferably between 4 h and 10 h.

According to one embodiment, the thermal cracking reaction takes place at atmospheric pressure or under slight pressure (maximum 0.2 MPa).

According to one embodiment, the product at the top of the cracker is recycled to the boiler of the dehydration column.

According to one embodiment, the product from the top of the cracker is mixed with the product from the top of the finishing column.

According to one embodiment, the bottom stream from the reactor (residue) obtained on conclusion of the thermal cracking operation has a dynamic viscosity of less than 1 Pa·s, preferably less than 10 Pa·s, measured at a temperature of 100° C., for example using a Brookfield “CAP 1000+” viscometer of cone/plate type.

According to one embodiment, polymerization inhibitors are used in at least one of the steps of the production process according to the invention. Polymerization inhibitors can be added at different locations, with the introduction of reactants or at the top of the distillation column, exchangers and condensers.

Mention may be made, as polymerization inhibitors which can be used, of for example phenothiazine (PTZ), hydroquinone (HQ), hydroquinone monomethyl ether (HQME), di-tert-butyl para-cresol (BHT), para-phenylenediamine, TEMPO (2,2,6,6-tetramethyl-1-piperidinyloxy), di-tert-butylcatechol, or TEMPO derivatives such as OH-TEMPO, manganese acetate, alone or mixtures thereof in any proportions, at contents in the reaction medium which may be between 50 ppm and 5000 ppm, optionally in the presence of depleted air, but generally in contents of between 150 ppm and 1000 ppm.

To make the inhibitors more effective, it is appropriate to inject oxygen, air or “depleted” air containing 7% O₂ at the bottom of the column. Preferably, the amount of oxygen injected corresponds to a content of 0.2% to 0.5% relative to the amount of organic vapor in the column.

According to one embodiment, said hydrothermal gasification equipment comprises a first reactor, a second reactor and a liquid gas separator.

According to one embodiment, the residue is injected as it is into the gasification and the water required for the hydrothermal treatment is injected elsewhere.

According to one embodiment, the residue is mixed with the water required for the hydrothermal treatment before introduction into the gasification.

According to the embodiment, the hydrothermal gasification is carried out at a temperature of 350-450° C. and a pressure of 25 MPa.

According to the embodiment, the hydrothermal gasification comprises a gasifier for separating the salt at the bottom and a gas and liquid mixture at the top.

According to one embodiment, the hydrothermal gasification comprises a separator for separating the salt under critical conditions, a gasifier and a liquid gas separator.

According to one embodiment, the hydrothermal gasification comprises a salt separator, a gasifier comprising a catalyst, and a liquid gas separator.

According to one embodiment, the concentration of residue/water+residue in the salt separator is between 10 g/l and 400 g/l.

According to one embodiment, the water used to carry out the hydrothermal gasification may be demineralized water, water from drilling, or weakly mineralized water.

According to one embodiment, the water leaving the gasifier, free of organic compounds, can advantageously be recycled to the feed of the separator or to the feed of the hydrolyzer.

According to one embodiment, the salts obtained and separated can be upgraded as fertilizers.

According to one embodiment, a proportion of 94% to 99% of the carbon introduced into the gasification is upgraded in the form of gas.

According to one embodiment, the gas resulting from the gasification is composed of 40-70% methane, 5-20% hydrogen and 20-40% carbon dioxide.

According to one embodiment, the gases can be further fractionated to isolate methane from the other compounds.

According to the process represented in FIG. 1, a gas reaction mixture 1 comprising acrylic acid obtained by gas-phase oxidation of a precursor of acrylic acid feeds a first distillation column 10. The gas reaction mixture comprising a water/acrylic acid weight ratio generally of between 0.3 and 2, preferably between 0.3 and 1.2, can be precooled before being subjected to dehydration in the dehydration column 10.

The reaction mixture comprises, in addition to water and acrylic acid, noncondensable light products such as nitrogen, oxygen, carbon monoxide and carbon dioxide, as well as various light or heavy by-products of different chemical natures which can be light aldehydes, such as acrolein, formaldehyde or acetaldehyde, heavy aldehydes, such as furfuraldehyde or benzaldehyde, light acids, such as formic acid, acetic acid or propionic acid, heavy acids, such as maleic acid, benzoic acid or 2-butenoic acid, and protoanemonin, a heavy compound of lactone type.

The dehydration column results in a top stream 2, at least a portion of which is condensed in a condenser 13 and returned to the dehydration column in reflux form 7 to absorb the acrylic acid, the other portion (stream 14) comprising the noncondensable light compounds being generally sent partially or completely to a purification device or recycled in part to other steps of the process for producing acrylic acid, preferably in a step located upstream of the reactor for the production of the reaction mixture 1.

The aim of the dehydration step is to remove, in a top stream, the bulk of the water present in the reaction mixture but also the noncondensable light compounds and the condensable light compounds. It generates a top stream 2 comprising the bulk of the water and of the light compounds, with acrylic acid and heavy compounds in a very small amount, and a bottom stream 15 depleted in light compounds comprising most of the acrylic acid with heavy by-products, and a content by weight of water generally of less than 10%, preferably of less than 7%.

A typical weight composition of the bottom stream 15 from the dehydration column essentially comprises acrylic acid (70-90%), acetic acid (2-20%), water (2-15%) and heavy by-products.

The dehydration column generally comprises from 5 to 50 theoretical plates, preferably from 20 to 30 theoretical plates.

Advantageously, the dehydration column operates at atmospheric pressure or slightly higher, up to an absolute pressure of 1.5×10⁵ Pa.

Advantageously, the temperature in the upper part of the dehydration column is at least 40° C., preferably between 40° C. and 80° C. The temperature of the bottom stream from the dehydration column preferably does not exceed 120° C.

The bottom stream 15 from the dehydration column is sent, at least in part (stream 3), to the top of a second distillation column 16, called a purification column or finishing column, wherein a top stream 8 and a bottom stream 9 are separated.

A portion 20 of the bottom liquid stream 15 from the dehydration column is sent to a heat exchanger 12, which can be a heater or a cooler, and reinjected into the dehydration column, so as to constitute a bottom recirculation loop. Preferably, the part 11 from the bottom loop is reinjected between the feed of the gas reaction mixture and the dehydration column top.

The remainder (stream 3) of the liquid stream 15 is sent as feed for the finishing column 16.

The finishing column 16 is generally a conventional distillation column comprising from 5 to 30 theoretical plates, preferably from 8 to 20 theoretical plates. This distillation column is combined, at the bottom, with at least one reboiler 17 and, at the top, with a condenser 19.

The temperature and the pressure in the column 16 are not critical, and can be determined in accordance with the distillation methods known from the prior art. However, preferably, the finishing column 16 operates at a pressure below atmospheric pressure, making it possible to operate at relatively low temperatures, thus preventing polymerization of the unsaturated products present, and minimizing the formation of heavy by-products.

Advantageously, the finishing column operates under an absolute pressure ranging from 5 kPa to approximately 60 kPa, the temperature of the top stream advantageously being between 40° C. and approximately 90° C., and the temperature of the bottom stream being between 60° C. and 120° C.

The top gas stream 8 from the finishing column is sent to the condenser 19, and the exiting liquid stream 4 is returned to the dehydration column, mixed with the stream from the bottom loop of the dehydration column. The top stream 8 comprises water and condensable light by-products.

The withdrawal sidestream 5 located in the first third of the bottom of the finishing column, preferably above the theoretical plate 3 starting from the bottom of the column, comprises technical-grade acrylic acid with a purity of >98.5%.

The stream 9 separated at the bottom of the finishing column comprises the bulk of the heavy by-products, in particular of the Michael addition products, such as 3-acryloyloxypropionic acid, maleic anhydride/acid, benzoic acid and also polymerization inhibitors. A typical weight composition of the bottom stream 9 essentially comprises acrylic acid (70-90%), polymerization inhibitors (0.5-2%) and heavy by-products (5-30%).

This stream 9 can be partially recycled to the bottom of the finishing column, or sent to a falling film evaporator via the line 6.

The evaporator 21 operates at a reduced pressure of 0.5 kPa to 60 kPa and in a temperature range of 50° C. to 150° C. After condensation and addition of stabilizer, the gas stream 22 essentially comprising acrylic acid is returned to the column one theoretical stage below the sidestream withdrawal.

Preferentially, the residue 25 also comprises an acrylic acid content of >10% and <40% in order to limit the viscosity thereof. This stream 25 and also water 34 are introduced under pressure into a reactor which makes it possible to hydrolyze for a time of between 1 h and 5 h under an autogenous pressure of 1a atmospheric pressure to 2 MPa in a temperature range extending from 100° C. to 170° C. This reactor can be a perfectly stirred reactor, a reactor equipped with an external recirculation loop and an exchanger, or a piston reactor. The stream 27 then feeds a thermal cracker.

This cracker 28 comprises a liquid gas separator, and an external recirculation loop fed by a tubular exchanger heated by steam having a pressure of between 1.5 and 3 MPa. The cracking time is between 1 h and 10 h at a pressure close to atmospheric pressure. After total condensation and addition of inhibitor, the top stream 32 is mixed with the stream 4 which returns to the dehydration column.

The residue 30 and water 37 are injected by means of two circuits via high pressure pumps into hydrothermal gasification equipment 33 in a temperature range of between 350° C. and 450° C. and at a pressure of 25 MPa. This equipment comprises:
a first reactor for separating the salt at the bottom of the reactor from the water and organic solution,
a second gasification reactor comprising a catalyst, which makes it possible to complete the conversion of the organic products to gas, and
a liquid gas separator which makes it possible to recover, at the bottom, an aqueous phase 35 which can be recycled to the inlet of the separator or to the inlet of the hydrolyzer, and a gas phase 36 rich in methane which can be upgraded to produce current, which can bring the energy necessary for the operation of the gasification but also for those of the reaction and purification line of this process or be exported elsewhere.

This hydrothermal gasification can be carried out in batch mode, or preferably in continuous mode.

The examples below illustrate the present invention without, however, limiting the scope thereof.

### EXPERIMENTAL SECTION

In the examples, the percentages are shown by weight, unless otherwise indicated, and the following abbreviations were used:
PTZ: Phenothiazine
AA: Acrylic acid
MA: Maleic acid
H₂O: Water
DiAA: Acrylic acid dimer
AA3: Acrylic acid trimer
Heavy compounds: Oligomers with a weight greater than AA3
HQ: Hydroquinone
ACOH: Acetic acid
HAA: Heavy acrylic acid compounds

### Examples

### Solvent-Free Pilot Purification Test

The characteristics of the solvent-free process are as follows:

Dehydration Column: diameter 300 mm
Number of theoretical stages: 22
   Finishing Column; diameter 300 mm
Number of theoretical stages: 17
Sidestream withdrawal: 14

The compositions obtained at the various points of the process are indicated in Table 1:

TABLE 1

| | Dehydration | Finishing | | Finishing |
|---|---|---|---|---|
| | column | column | Sidestream | column |
| | bottom | bottom | withdrawal | top |
| AA (%) | 71.65 | 86.54 | 98.95 | 64.8 |
| ACOH (%) | 11.9 | 0.0022 | 0.0084 | 15 |
| DiAA (%) | 0.84 | 10.94 | 0.77 | 1.42 |
| H₂O (%) | 14 | 0.047 | 0.094 | 17.22 |
| MA (ppm) | 980 | 6300 | 124 | 20 |
| HQ (ppm) | 845 | 11130 | 400 | 44 |
| PTZ (ppm) | 110 | 5000 | 750 | 50 |

The column bottom contains about 11% of DiAA which will be concentrated on a film evaporator.

### Concentration of the Column Bottom on a Film Evaporator

The concentration of the column bottom was simulated on Aspen.

The operating conditions and compositions are given in Table 2 below:

TABLE 2

| | Units | Feed | BOTTOM | TOP |
|---|---|---|---|---|
| Temperature | ° C. | 50 | 130 | 130 |
| Pressure | MPa | 0.1 | 0.04 | 0.04 |
| Weight flow rate | kg/hr | 100.0 | 19.7 | 80.3 |
| Weight fraction | | | | |
| AA | | 0.87 | 0.40 | 0.98 |
| DIAA | | 0.11 | 0.49 | 0.02 |
| HQ | | 0.011 | 0.054 | 0.0005 |
| PTZ | | 0.005 | 0.025 | 1.63 × 10⁻⁵ |
| MA | | 0.006 | 0.031 | 0.0001 |

The concentration of the bottom product by evaporation is efficient. In fact, there is a decrease in the AA content in the bottom. Conversely, this evaporation, which generates at the top an acrylic acid with a purity of 98%, is not efficient enough for this product to be mixed with the technical-grade acrylic acid obtained at the sidestream withdrawal and will therefore have to be recycled to the column.

Thermal Cracking with or without Prior Hydrolysis

This example corresponds to examples 1 and 2 of application FR 2206330 and shows the advantage of treating the bottom of the evaporator by cracking preceded by hydrolysis in terms of the efficiency of this treatment. The results are presented in Table 3.

It should also be noted that the top product will therefore be composed of about 70% AA and 30% water, which is very close to the composition of the top of the finishing column and makes mixing these two fluids very easy.

TABLE 3

| | With hydrolysis | | Without hydrolysis | |
|---|---|---|---|---|
| Test No. | 1 | | 2 | |
| Hydrolysis - temperature | 150° | C. | — | |
| Hydrolysis residence time | 1 | h | — | |
| Water/HAA ratio | 0.5 | | — | |
| Cracking temperature | 173° | C. | 183° | C. |
| Cracking residence time | 10 | h | 10 | h |

| | | |
|---|---|---|
| Useful recovery rate | 68.95% | 29.2% |
| Overall cracking rate | 94.85% | 45.1% |
| Viscosity of the residue | 0.785 | 1.034 |
| in Pa · s @ 100° C. | | |

### Hydrothermal Gasification

Hydrothermal gasification will be illustrated by a very similar case of Michael adducts, namely those of heavy butyl acrylate compounds.

The heavy ABU compound mixture is composed of
Butanol<0.1%
Butyl acrylate (5-10%)
Butyl hydroxypropionate (BHP): 1-3%
Butyl butoxypropionate (BPB) 70-80%
Butyl acryloxypropionate (AA/ABU) 4-6%
Dibutyl maleate: 2-5%
Phenothiazine: 1-3%.

33 g/h of heavy ABU compounds and 970 g/h of water are introduced via two different pipes into a separator and a catalytic reactor, both operating at 400° C. and 25 MPa. After 6 hours of testing under stabilized conditions, the heavy ABU compounds are converted into a gas mixture having the following volume composition: 51% CH₄; 34% CO₂ and 19% H₂. The amount of energy of this gas corresponds to 7096 kWh/tonne ABU. The amount of TOC (Total Organic Carbon) is <1 mg/l.

1. A process for producing technical-grade (meth)acrylic acid in the absence of organic solvent, from a gas reaction mixture comprising (meth)acrylic acid obtained by gas-phase oxidation of a precursor of the (meth)acrylic acid, comprising the following steps:
a. a step of dehydrating said gas reaction mixture in a first distillation column called a dehydration column, resulting in a top stream, wherein at least a portion of the top stream is condensed and returned to the dehydration column in a reflux form, and in a bottom stream, wherein at least a portion of the bottom stream is returned to a lower part of the dehydration column to form a recirculation loop;
b. a step of distilling at least a portion of said bottom stream from the dehydration column in a second distillation column, called a finishing column, making it possible to separate a bottom stream containing heavy compounds, a top stream containing light compounds, wherein at least a portion of the top stream containing light compounds is returned to the dehydration column, and a withdrawal sidestream of technical-grade (meth)acrylic acid;
c. a step of concentrating said bottom stream from the finishing column in an evaporator resulting in an evaporator bottom stream concentrating the Michael adducts, and an evaporator top stream comprising (meth)acrylic acid which is returned to the finishing column;
d. a step of hydrolyzing said evaporator bottom stream, in the presence of water, in a hydrolyzer, resulting in production of a stream of hydrolyzed products;
e. a step of thermally cracking said stream of hydrolyzed products in a cracker, resulting in production of a cracker top stream which is recycled to the dehydration column, and a cracker bottom residue; and
f. a step of hydrothermally treating said cracker bottom residue, in the presence of water, carried out in a hydrothermal gasification equipment, resulting in production, from a top of the hydrothermal gasification equipment, of methane, hydrogen and CO2 gases and, from a bottom of the hydrothermal gasification equipment, of solid residues and water.

2. The process as claimed in claim 1, wherein the evaporator is placed at the bottom of the finishing column and is a film evaporator working at a reduced pressure of 0.5 to 100 kPa.

3. The process as claimed in claim 1, wherein the evaporator top stream is recycled to the bottom of the finishing column, below the sidestream withdrawal line.

4. The process as claimed in claim 1, wherein a temperature of the hydrolyzer varies between 80° C. and 200° C.

5. The process as claimed in claim 1, wherein a weight ratio of water to the Michael adducts in the hydrolyzer ranges from 0.1 to 1.3, limits included.

6. The process as claimed in claim 1, wherein the thermal cracking is performed on acrylic acid adducts.

7. The process as claimed in claim 1, wherein the thermal cracking is performed on a mixture of adducts of acrylic acid and of acrylic esters.

8. The process as claimed in claim 1, further comprising a step of mixing the cracker top stream with the top stream from the finishing column.

9. The process as claimed in claim 1, wherein the hydrothermal gasification comprises a separator for separating a salt under critical conditions, a gasifier and a liquid gas separator.

10. The process as claimed in claim 9, wherein the concentration of residue/water+residue in said separator is between 10 g/l and 400 g/l.

11. The process as claimed in claim 1, wherein the gasification generates a gas comprising 40% to 70% methane, 5-20% hydrogen and 20-40% carbon dioxide.

12. The process as claimed in claim 9, wherein water leaving the gasifier, free of organic compounds, is recycled to the separator or the hydrolyzer.
